(19) United States
(12) Patent Application Publication
(10) Pub. No.: US 2025/0235291 A1
(43) Pub. Date: Jul. 24, 2025

(54) Step Device

(71) Applicant: Crystal Nicole Gardner, Boerne, TX (US)

(72) Inventor: Crystal Nicole Gardner, Boerne, TX (US)

(21) Appl. No.: 18/790,381

(22) Filed: Jul. 31, 2024

Related U.S. Application Data
(60) Provisional application No. 63/622,256, filed on Jan. 18, 2024.

(51) Int. Cl. A61B 90/60; A61G 7/05

(52) U.S. Cl. CPC A61B 90/60; A61G 7/05

(57) ABSTRACT

A step device is disclosed that assists healthcare workers regardless of size or weight in providing procedures within a hospital or other medical facility. The step device comprises a step component that is configured in a rectangular shape and is sized to retain two feet of a user with ease. A leg is secured with a hinge to each of the four corners of the step component. The four legs act to fold up into the underside of the step component when not in use. A support member is secured to the opposing right and left sides of the step component. Both support members then secure to an underside of the hospital bed with a hinge, allowing the support members to fold the step component up under the bed when not in use and extending the step component down to the floor for use.

### CROSS-REFERENCE TO RELATED APPLICATION

The present application claims priority to, and the benefit of, U.S. Provisional Application No. 63/622,256, which was filed on Jan. 18, 2024, and is incorporated herein by reference in its entirety.

### FIELD OF THE INVENTION

The present invention relates generally to the field of step devices. More specifically, the present invention relates to a step device for assisting healthcare workers when they need to perform different procedures on a patient in a hospital bed. Accordingly, the present disclosure makes specific reference thereto. Nonetheless, it is to be appreciated that aspects of the present invention are also equally applicable to other like applications, devices and methods of manufacture.

### BACKGROUND

By way of background, this invention relates to improvements in step devices. Generally, medical professionals who are shorter may have trouble accessing a patient's elevated hospital bed. Additionally, using a step stool during surgical procedures can be frustrating and potentially dangerous as the step stool may fall over.

Further, time is crucial when performing different medical procedures like CPR and taking time to place a stepstool down, climb up the stool, and then begin the procedure can mean the difference between life and death. Users may not have time to retrieve a step stool and stand on it before a procedure needs to start.

Accordingly, there is a demand for an improved step device that assists healthcare workers when they need to perform different procedures on a patient in a hospital bed. More particularly, there is a demand for a step device that offers a way for medical professionals to perform lifesaving procedures quickly, regardless of their height.

Therefore, there exists a long felt need in the art for a step device that provides users with a means for assisting healthcare workers when they need to perform different procedures on a patient in a hospital bed. There is also a long felt need in the art for a step device that allows users to press a button to extend the step from the left and/or right side of the hospital bed for easy access. Further, there is a long felt need in the art for a step device that folds out from underneath the bed and enables the medical practitioner to quickly step up and view the patient in the bed. Moreover, there is a long felt need in the art for a device that eliminates the need to search for and use independent step stools when working with a patient. Further, there is a long felt need in the art for a step device that offers a way for medical professionals to perform lifesaving procedures quickly, regardless of their height. Finally, there is a long felt need in the art for a step device that is durable and can accommodate up to 700 lbs.

The subject matter disclosed and claimed herein, in one embodiment thereof, comprises a step device. The device assists all healthcare workers regardless of size or weight in providing procedures within a hospital or other various medical facilities. The step device comprises a step component that is configured in a rectangular shape and is sized to retain two feet of a user with ease. A leg is secured with a hinge to each of the four corners of the step component. The four legs act to fold up into the underside of the step component when not in use. A support member is secured to the opposing right and left sides of the step component. Both support members then secure to an underside of the hospital bed with a hinge, allowing the support members to fold the step component up under the bed when not in use and extending the step component down to the floor for use. Typically, the step component, legs, and the support members are spring-loaded, such that when the step component is extended down to the floor, it is in an un-loaded position. In a spring-loaded position, the step component and legs are folded up under the hospital bed and locked in position with a latch or hook, or other securing means. A user would then push a button, which releases the latch or hook, which allows the step component and legs to extend down toward the floor for use.

In this manner, the step device of the present invention accomplishes all of the forgoing objectives and provides users with a device that offers a way for medical professionals to perform procedures regardless of their height. The device is a step component that folds up under the hospital bed when not in use. The device can be manufactured of a plastic or metal material.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a step device. The device assists all healthcare workers regardless of size or weight in providing procedures within a hospital or other various medical facilities. The step device comprises a step component that will fold out from under a hospital bed, as needed. The step component comprises four legs, which fold up into the underside of the step component when not in use. Support members attached to the underside of a hospital bed, are also secured to the step component. The support members fold the step component up under the bed when not in use and extend the step component down to the floor for use.

In one embodiment, the step device provides a way for medical professionals to perform lifesaving procedures, regardless of their height. The device ensures that medical professionals and other users do not need to search for step stools when working with a patient. The step device can be folded out from either side of a hospital bed for use. A user would engage the step device to quickly step up and view the patient in the hospital bed.

In one embodiment, the step device comprises a step component that is configured in a rectangular shape, but can be any suitable shape as is known in the art, such as oval, square, etc. Further, the step component is typically sized to retain a user's two feet with ease but can be any suitable shape and size as is known in the art, depending on the needs and/or wants of a user. Additionally, the step component comprises a top side, an underside, opposing side edges, and opposing front and back edges.

In one embodiment, the top side of the step component comprises tread sections or other such gripping texture areas to prevent users from slipping and to provide a non-slip grip for a user when using the step device.

In one embodiment, a leg is secured with a hinge to each of the four corners of the step component. The legs are secured to the underside of the step component via any suitable securing means as is known in the art. The four legs act to fold up into the underside of the step component when not in use via the hinges. The legs can be any suitable length, depending on the needs and/or wants of a user.

In one embodiment, a support member is secured to the opposing right and left sides of the step component. Both support members then secure to an underside of the hospital bed with a hinge, allowing the support members to fold the step component up under the bed when not in use and extending the step component down to the floor for use. The support members can be any suitable length as is known in the art, depending on the width of the hospital bed, and the needs and/or wants of a user. Typically, the support members are secured to the step component via bolts, screws, rivets, etc., or any other suitable securing means as is known in the art.

In one embodiment, the support members can be secured, such that the step component extends from the right side of the hospital bed. In another embodiment, the support members can be secured, such that the step component extends from the left side of the hospital bed. In yet another embodiment, two sets of support members can be secured to the underside of a hospital bed, such that a step component can be extended from the right side and the left side of the hospital bed, as needed. Thus, users can choose to extend the step device from the left side, the right side, or both sides of the hospital bed, as needed to perform different procedures.

In one embodiment, the step device is durable enough to accommodate up to approximately 700 pounds. Thus, the step device ensures that medical professionals can maintain their balance and stability no matter their weight or size, and even if they need to apply pressure to the patient or utilize heavy tools during the procedures being performed.

In one embodiment, typically, the step component, legs, and the support members are spring-loaded, such that when the step component is extended down to the floor, it is in an un-loaded position. In a spring-loaded position, the step component and legs are folded up under the hospital bed and locked in position with a latch or hook, or other securing means. A user would then push a button, which releases the latch or hook, which allows the step component and legs to extend down toward the floor for use. Typically, the legs utilize a pin bar mechanism or other suitable mechanism to fold up with the step component and then un-fold when needed.

In one embodiment, the support members of the step device comprise an electric motor in communication with the hospital bed's electricity and power. Thus, the hospital bed's electricity is utilized to actuate the motor. Specifically, actuating the motor, moves the step component up and down via the press of a button.

In one embodiment, the step device and its components are manufactured of metal, such as stainless steel or aluminum, or from heat-sealable plastic or polymers, such as polypropylene or acrylonitrile-butadiene-styrene (ABS), acrylic, polycarbonate, polyethylene, polyethylene terephthalate, polyvinyl chloride, polystyrene, etc. Generally, the step device is also manufactured from a material that is water resistant or waterproof or comprises a coating that is water resistant or waterproof. Furthermore, the device can be made of antibacterial or antimicrobial material or comprises a coating that is antibacterial or antimicrobial.

In yet another embodiment, the step device comprises a plurality of indicia.

In yet another embodiment, a method of easily and quickly elevating a medical professional over a hospital bed is disclosed. The method includes the steps of providing a step device comprising a step component, legs, and support members. The method also comprises securing the support members to an underside of a hospital bed. Further, the method comprises folding the step component up under the hospital bed and securing it in place. Finally, the method comprises pushing a button to release the step component down for use.

Numerous benefits and advantages of this invention will become apparent to those skilled in the art to which it pertains, upon reading and understanding the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one embodiment of the step device of the present invention in accordance with the disclosed architecture;

FIG. 2 illustrates a perspective view of one embodiment of the step device of the present invention showing the step component extended from a right side of a hospital bed in accordance with the disclosed architecture;

FIG. 3 illustrates a perspective view of one embodiment of the step device of the present invention showing the step component extended from a left side of a hospital bed in accordance with the disclosed architecture;

FIG. 4 illustrates a perspective view of one embodiment of the step device of the present invention showing a user elevated off the ground via the step device in accordance with the disclosed architecture;

FIG. 5 illustrates a perspective view of one embodiment of the step device of the present invention in use in accordance with the disclosed architecture; and

FIG. 6 illustrates a flowchart showing the method of easily and quickly elevating a medical professional over a hospital bed in accordance with the disclosed architecture.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long felt need in the art for a step device that provides users with a means for assisting healthcare workers when they need to perform different procedures on a patient in a hospital bed. There is also a long felt need in the art for a step device that allows users to press a button to extend the step from the left and/or right side of the hospital bed for easy access. Further, there is a long felt need in the art for a step device that folds out from underneath the bed and enables the medical practitioner to quickly step up and view the patient in the bed. Moreover, there is a long felt need in the art for a device that eliminates the need to search for and use independent step stools when working with a patient. Further, there is a long felt need in the art for a step device that offers a way for medical professionals to perform lifesaving procedures quickly, regardless of their height. Finally, there is a long felt need in the art for a step device that is durable and can accommodate up to 700 lbs.

The present invention, in one exemplary embodiment, is a novel step device. The step device comprises a step component that is configured in a rectangular shape and is sized to retain two feet of a user with ease. A leg is secured with a hinge to each of the four corners of the step component. The four legs act to fold up into the underside of the step component when not in use. A support member is secured to the opposing right and left sides of the step component. Both support members then secure to an underside of the hospital bed with a hinge, allowing the support members to fold the step component up under the bed when not in use and extending the step component down to the floor for use. Typically, the step component, legs, and the support members are spring-loaded. The present invention also includes a novel method of easily and quickly elevating a medical professional over a hospital bed. The method includes the steps of providing a step device comprising a step component, legs, and support members. The method also comprises securing the support members to an underside of a hospital bed. Further, the method comprises folding the step component up under the hospital bed and securing it in place. Finally, the method comprises pushing a button to release the step component down for use.

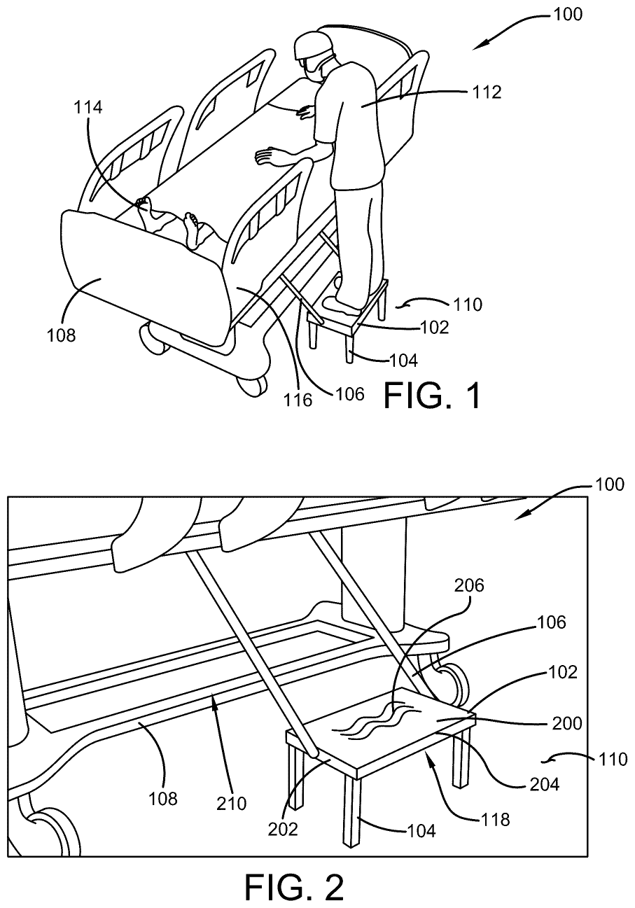

Referring initially to the drawings, FIG. 1 illustrates a perspective view of one embodiment of the step device 100 of the present invention. In the present embodiment, the step device 100 is an improved step device 100 that assists healthcare workers 112 when performing procedures on patients 114 in a hospital bed 108. Specifically, the step device 100 comprises a step component 102 that will fold out from under a hospital bed 108, as needed. The step component 102 comprises four legs 104, which fold up into the underside 118 of the step component 102 when not in use. Support members 106 attached to the underside 116 of a hospital bed 108, are also secured to the step component 102. The support members 106 fold the step component 102 up under the bed 108 when not in use and extend the step component 102 down to the floor 110 for use.

Generally, the step device 100 provides a way for medical professionals 112 to perform lifesaving procedures, regardless of their height. The device 100 ensures that medical professionals 112 and other users do not need to search for step stools when working with a patient 114. The step device 100 can be folded out from either side of a hospital bed 108 for use. A user 112 would engage the step device 100 to quickly step up and view the patient 114 in the hospital bed 108.

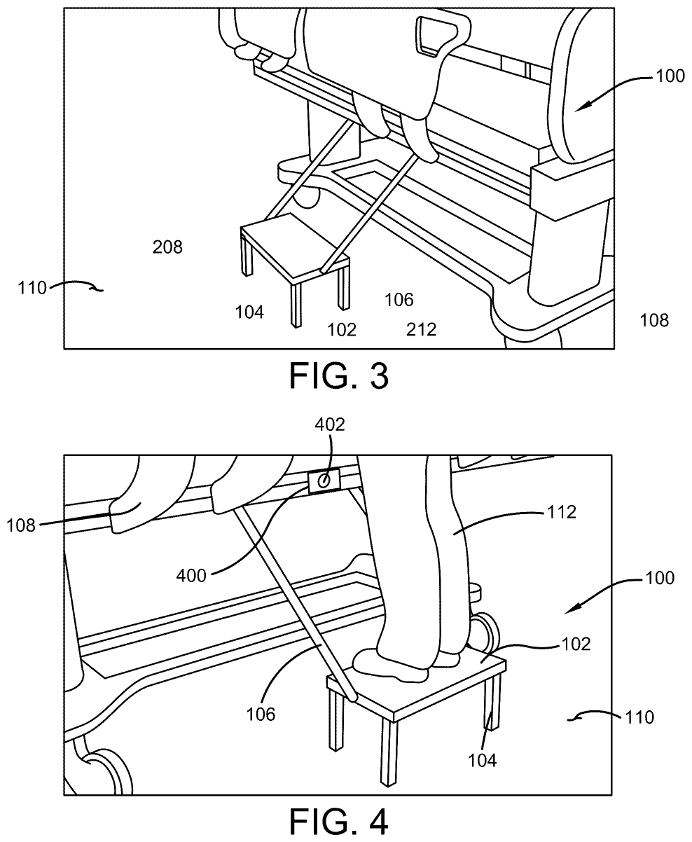

As shown in FIGS. 2-3, the step device 100 comprises a step component 102 that is configured in a rectangular shape, but can be any suitable shape as is known in the art, such as oval, square, etc. Further, the step component 102 is typically sized to retain a user's two feet with ease but can be any suitable shape and size as is known in the art, depending on the needs and/or wants of a user 112. Additionally, the step component 102 comprises a top side 200, an underside 118, opposing side edges 202, and opposing front and back edges 204.

Further, the top side 200 of the step component 102 comprises tread sections 206 or other such gripping texture areas to prevent users 112 from slipping and to provide a non-slip grip for a user 112 when using the step device 100.

Additionally, a leg 104 is secured with a hinge 208 to each of the four corners 210 of the step component 102. The legs 104 are secured to the underside 118 of the step component 102 via any suitable securing means as is known in the art. The four legs 104 act to fold up into the underside 118 of the step component 102 when not in use via the hinges 208. The legs 104 can be any suitable length, depending on the needs and/or wants of a user 112.

Furthermore, a support member 106 is secured to the opposing right and left sides 202 of the step component 102. Both support members 106 then secure to an underside 116 of the hospital bed 108 with a hinge, allowing the support members 106 to fold the step component 102 up under the bed 108 when not in use and extending the step component 102 down to the floor 110 for use. The support members 106 can be any suitable length as is known in the art, depending on the width of the hospital bed 108, and the needs and/or wants of a user 112. Typically, the support members 106 are secured to the step component 102 via bolts, screws, rivets, etc., or any other suitable securing means as is known in the art.

Additionally, the support members 106 can be secured, such that the step component 102 extends from the right side 210 of the hospital bed 108. In another embodiment, the support members 106 can be secured, such that the step component 102 extends from the left side 212 of the hospital bed 108. In yet another embodiment, two sets of support members 106 can be secured to the underside 116 of a hospital bed 108, such that a step component 102 can be extended from the right side 210 and the left side 212 of the hospital bed 108, as needed. Thus, users 112 can choose to extend the step device 100 from the left side 212, the right side 210, or both sides 210, 212 of the hospital bed 108, as needed to perform different procedures.

As shown in FIG. 4, the step device 100 is durable enough to accommodate up to approximately 700 pounds. Thus, the step device 100 ensures that medical professionals 112 can maintain their balance and stability no matter their weight or size, and even if they need to apply pressure to the patient 114 or utilize heavy tools during the procedures being performed.

In one embodiment, typically, the step component 102, legs 104, and the support members 106 are spring-loaded, such that when the step component 102 is extended down to the floor 110, it is in an un-loaded position. In a spring-loaded position, the step component 102 and legs 104 are folded up under the hospital bed 108 and locked in position with a latch 400 or hook, or other securing means. A user 112 would then push a button 402, which releases the latch 400 or hook, which allows the step component 102 and legs 104 to extend down toward the floor 110 for use. Typically, the legs 104 utilize a pin bar mechanism or other suitable mechanism to fold up with the step component 102 and then un-fold when needed.

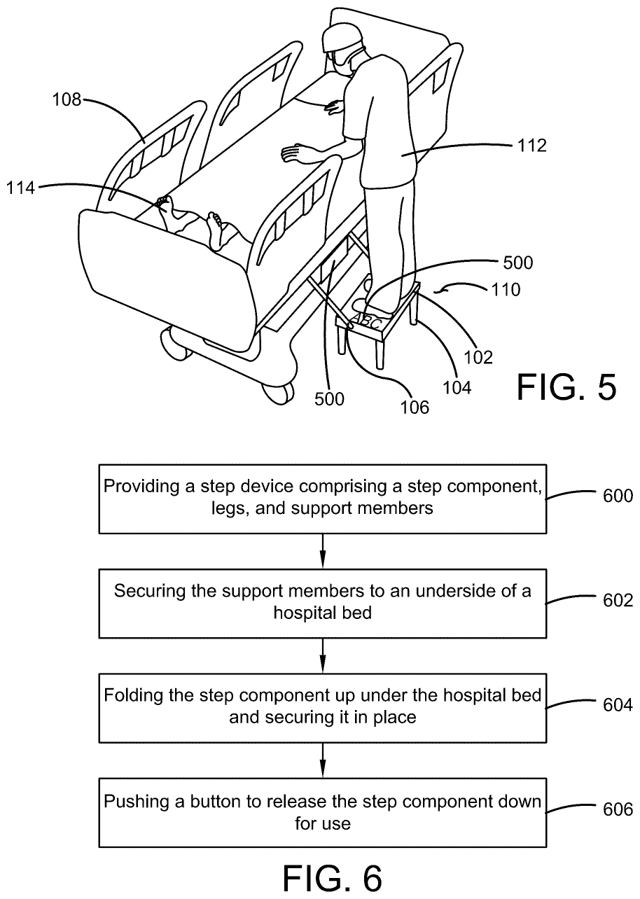

As shown in FIG. 5, in another embodiment, the support members 106 of the step device 100 comprise an electric motor 502 in communication with the hospital bed's electricity and power. Thus, the hospital bed's electricity is utilized to actuate the motor 502. Specifically, actuating the motor 502, moves the step component 102 up and down via the press of a button 402.

Further, the step device 100 and its components are manufactured of metal, such as stainless steel or aluminum, or from heat-sealable plastic or polymers, such as polypropylene or acrylonitrile-butadiene-styrene (ABS), acrylic, polycarbonate, polyethylene, polyethylene terephthalate, polyvinyl chloride, polystyrene, etc. Generally, the step device 100 is also manufactured from a material that is water resistant or waterproof or comprises a coating that is water resistant or waterproof. Furthermore, the device 100 can be made of antibacterial or antimicrobial material or comprises a coating that is antibacterial or antimicrobial.

In yet another embodiment, the step device 100 comprises a plurality of indicia 500. The step component 102 of the device 100 may include advertising, a trademark, or other letters, designs, or characters, printed, painted, stamped, or integrated into the step component 102, or any other indicia 500 as is known in the art. Specifically, any suitable indicia 500 as is known in the art can be included, such as but not limited to, patterns, logos, emblems, images, symbols, designs, letters, words, characters, animals, advertisements, brands, etc., that may or may not be step stool, hospital bed, or brand related.

FIG. 6 illustrates a flowchart of the method of easily and quickly elevating a medical professional over a hospital bed. The method includes the steps of at 600, providing a step device comprising a step component, legs, and support members. The method also comprises at 602, securing the support members to an underside of a hospital bed. Further, the method comprises at 604, folding the step component up under the hospital bed and securing it in place. Finally, the method comprises at 606, pushing a button to release the step component down for use.

Certain terms are used throughout the following description and claims to refer to particular features or components. As one skilled in the art will appreciate, different users may refer to the same feature or component by different names. This document does not intend to distinguish between components or features that differ in name but not structure or function. As used herein “step device” and “device” are interchangeable and refer to the step device 100 of the present invention.

Notwithstanding the forgoing, the step device 100 of the present invention can be of any suitable size and configuration as is known in the art without affecting the overall concept of the invention, provided that it accomplishes the above stated objectives. One of ordinary skill in the art will appreciate that the step device 100 as shown in FIGS. 1-6 is for illustrative purposes only, and that many other sizes and shapes of the step device 100 are well within the scope of the present disclosure. Although the dimensions of the step device 100 are important design parameters for user convenience, the step device 100 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A step device that assists healthcare works when performing procedures on patients in a hospital bed, the step device comprising:
a step component;
a plurality of legs; and
a pair of support members;
wherein the plurality of legs are secured to the step component;
wherein the pair of support members are secured to an underside of a hospital bed and then to the step component;
wherein the pair of support members fold the step component up under the hospital bed when not in use; and
further wherein the pair of support members extend the step component down to a floor for use.

2. The step device of claim 1, wherein the step component is configured in a rectangular shape, and is sized to retain a user's two feet with ease.

3. The step device of claim 2, wherein the step component comprises a top side, an underside, opposing side edges, and opposing front and back edges.

4. The step device of claim 3, wherein the top side of the step component comprises tread sections to prevent users from slipping.

5. The step device of claim 4, wherein a leg is secured with a hinge to each of four corners of an underside of the step component.

6. The step device of claim 5, wherein the legs fold up into the underside of the step component when not in use via the hinges.

7. The step device of claim 6, wherein the pair of support members are secured to the opposing side edges of the step component.

8. The step device of claim 7, wherein the pair of support members are secured to the underside of the hospital bed with a hinge, allowing the pair of support members to fold and extend the step component.

9. The step device of claim 8, wherein the step device is durable enough to accommodate up to approximately 700 pounds of weight from a user.

10. The step device of claim 9, wherein the step component, the plurality of legs, and the pair of support members are spring-loaded, such that when the step component is extended down to the floor, it is in an un-loaded position.

11. The step device of claim 10, wherein in a spring-loaded position, the step component and the plurality of legs are folded up under the hospital bed and locked in position with a latch.

12. The step device of claim 11 further comprising a button which the user would push to release the latch to deploy the step component and the plurality of legs toward the floor for use.

13. A step device that assists healthcare works when performing procedures on patients in a hospital bed, the step device comprising:
a step component configured in a rectangular shape, and sized to retain a user's two feet with ease;
a plurality of legs with a leg secured with a hinge to each of four corners of the step component; and
a pair of support members secured to opposing side edges of the step component; and
wherein the step device is durable enough to accommodate up to approximately 700 pounds of weight from a user;
wherein the plurality of legs are secured to the step component and fold up into an underside of the step component when not in use via the hinges;
wherein the pair of support members are secured to an underside of a hospital bed via a hinge;
wherein the pair of support members fold the step component up under the hospital bed when not in use; and
further wherein the pair of support members extend the step component down to a floor for use.

14. The step device of claim 13, wherein the pair of support members comprise an electric motor in communication with the hospital bed's power, which actuates the electric motor to move the step component up and down via a press of a button.

15. The step device of claim 13, wherein the step device is manufactured from a material that is water resistant or waterproof and antibacterial or antimicrobial.

16. The step device of claim 13, wherein the step component extends from a right side of the hospital bed.

17. The step device of claim 13, wherein the step component extends from a left side of the hospital bed.

18. The step device of claim 13, wherein a step component extends from a right side and a left side of the hospital bed.

19. The step device of claim 13 further comprising a plurality of indicia.

20. A method of easily and quickly elevating a medical professional over a hospital bed, the method comprising the following steps:
providing a step device comprising a step component, legs, and support members;
securing the support members to an underside of a hospital bed;
folding the step component up under the hospital bed and securing it in place; and
pushing a button to release the step component down for use.
